(19) United States
(12) Patent Application Publication
(10) Pub. No.: US 2024/0345062 A1
(43) Pub. Date: Oct. 17, 2024

(54) DETECTING HYDROCARBON FUELS IN LUBRICATION OILS

(71) Applicants: Saudi Arabian Oil Company, Dhahran (SA); King Abdullah University of Science and Technology, Thuwal (SA)

(72) Inventors: Maha Nour, Thuwal (SA); Khawlah Kharashi, Thuwal (SA); Khaled Nabil Salama, Thuwal (SA); Abdullah Hassan Bukhamsin, Thuwal (SA); Esraa Fakeih, Thuwal (SA); Sumana Bhattacharjee, Thuwal (SA)

(21) Appl. No.: 18/298,776

(22) Filed: Apr. 11, 2023

(51) Int. Cl. G01N 33/28; G01N 27/00

(52) U.S. Cl. CPC G01N 33/2888; G01N 27/002

(57) ABSTRACT

A fuel detection film includes a cyclic olefin copolymer that is insoluble in acyclic saturated hydrocarbons. The fuel detection film has a porous structure that defines a plurality of pores. The plurality of pores are configured to allow flow of a lubrication oil through the fuel detection film via the plurality of pores. A housing is configured to couple to a pipe flowing the lubrication oil. The housing defines a slot. The slot is configured to hold the fuel detection film. The fuel detection film, while held by the slot of the housing that is coupled to the pipe as the lubrication oil flows in the pipe, is configured to at least partially dissolve in a presence of an aromatic hydrocarbon in the lubrication oil flowing through the fuel detection film.

### TECHNICAL FIELD

This disclosure relates to detection of hydrocarbon fuels in lubrication oils.

### BACKGROUND

Fuel smuggling is unfortunately a lucrative illegal act that is committed in various parts of the world. In some cases, fuels are transported to neighboring countries through illegal means. In some cases, fuels are diluted to increase volume in generating an increased profit. Sometimes petroleum products are diluted at the midstream level and transported downstream for sale to avoid detection of such dilution. In other cases, fuels are mixed with and hidden in other products to avoid detection of smuggling.

### SUMMARY

This disclosure describes technologies relating to devices that can be used to detect hydrocarbon fuels in lubrication oils. Certain aspects of the subject matter described can be implemented as an assembly. The assembly includes a fuel detection film and a housing. The fuel detection film includes a cyclic olefin copolymer that is insoluble in acyclic saturated hydrocarbons. The fuel detection film has a porous structure that defines a plurality of pores. The plurality of pores are configured to allow flow of a lubrication oil through the fuel detection film via the plurality of pores. The housing is configured to couple to a pipe flowing the lubrication oil. The housing defines a slot. The slot is configured to hold the fuel detection film. The fuel detection film, while held by the slot of the housing that is coupled to the pipe as the lubrication oil flows in the pipe, is configured to at least partially dissolve in a presence of an aromatic hydrocarbon in the lubrication oil flowing through the fuel detection film.

This, and other aspects, can include one or more of the following features. The assembly can include a multimeter. The multimeter can be coupled to the fuel detection film at two locations on the fuel detection film. The multimeter can be configured to apply a current across the fuel detection film. The multimeter can be configured to measure a resistance of the fuel detection film at least based on a voltage drop exhibited by the current applied across the fuel detection film in response to the current applied across the fuel detection film. A decrease in the resistance of the fuel detection film can indicate at least partial dissolution of the fuel detection film and the presence of the aromatic hydrocarbon in the lubrication oil. The housing can include a first portion and a second portion coupled together. The slot can be defined between the first portion and the second portion. The first portion and the second portion can be made of silicon, glass, quartz, polymethyl methacrylate, or any combinations of these. The fuel detection film can have a thickness in a range of from about 5 micrometers (μm) to about 500 μm.

Certain aspects of the subject matter described can be implemented as a system. The system includes a pipe, a housing, a fuel detection film, and a multimeter. The pipe defines a first inner bore that is configured to flow a lubrication oil. The housing is coupled to the pipe. The housing defines a second inner bore and a cross-sectional slot. The second inner bore of the housing is aligned with the first inner bore of the pipe for flow of the lubrication oil. The fuel detection film is disposed within the cross-sectional slot of the housing. The fuel detection film spans across the second inner bore of the housing. The fuel detection film includes a cyclic olefin copolymer that is insoluble in acyclic saturated hydrocarbons. The fuel detection film has a porous structure that defines a plurality of pores. The plurality of pores are configured to allow flow of the lubrication oil through the fuel detection film via the plurality of pores. The fuel detection film is configured to at least partially dissolve in a presence of an aromatic hydrocarbon in the lubrication oil flowing through the fuel detection film. The multimeter is coupled to the fuel detection film at two locations on the fuel detection film. The multimeter is configured to apply a current across the fuel detection film. The multimeter is configured to measure a resistance of the fuel detection film at least based on a voltage drop exhibited by the current applied across the fuel detection film. A change in the resistance of the fuel detection film indicates at least partial dissolution of the fuel detection film and the presence of the aromatic hydrocarbon in the lubrication oil.

This, and other aspects, can include one or more of the following features. The housing can include a first portion and a second portion coupled together. The cross-sectional slot can be defined between the first portion and the second portion. The first portion and the second portion can be made of silicon, glass, quartz, polymethyl methacrylate, or any combinations of these. The fuel detection film can have a thickness in a range of from about 5 μm to about 500 μm.

Certain aspects of the subject matter described can be implemented as a method. A lubrication oil is flowed through a fuel detection film. The fuel detection film is coupled to a pipe. The fuel detection film includes a cyclic olefin copolymer that is insoluble in acyclic saturated hydrocarbons. The fuel detection film has a porous structure defining a plurality of pores. The plurality of pores are configured to allow flow of the lubrication oil through the fuel detection film via the plurality of pores. The fuel detection film is configured to at least partially dissolve in a presence of an aromatic hydrocarbon in the lubrication oil flowing through the fuel detection film. It is detected whether the fuel detection film has at least partially dissolved in response to the lubrication oil flowing through the fuel detection film. At least partial dissolution of the fuel detection film in response to the lubrication oil flowing through the fuel detection film indicates the presence of an aromatic hydrocarbon in the lubrication oil.

This, and other aspects, can include one or more of the following features. A current can be applied across the fuel detection film as the lubrication oil flows through the fuel detection film. A resistance of the fuel detection film can be measured at least based on a voltage drop exhibited by the current applied across the fuel detection film in response to the current applied across the fuel detection film. A multimeter can be coupled to the fuel detection film at two locations on the fuel detection film. The multimeter can apply the current across the fuel detection film and measures the resistance of the fuel detection film. Detecting whether the fuel detection film has at least partially dissolved can include detecting a change in the resistance of the fuel detection film. A decrease in the resistance of the fuel detection film can indicate at least partial dissolution of the fuel detection film and the presence of the aromatic hydrocarbon in the lubrication oil. The fuel detection film can be disposed within a cross-sectional slot of a housing that is coupled to the pipe. The housing can define a first inner bore that is aligned with a second inner bore of the pipe through which the lubrication oil flows. The fuel detection film can span across the first inner bore of the housing. The housing can include a first portion and a second portion coupled together. The cross-sectional slot can be defined between the first portion and the second portion. The fuel detection film can be sandwiched between the first portion and the second portion. The fuel detection film can be disposed within the cross-sectional slot of the housing. The first portion and the second portion can be coupled together, such that the fuel detection film is sandwiched between the first portion and the second portion, and the fuel detection film spans across the first inner bore of the housing. The housing can be coupled to the pipe, such that the first inner bore of the housing is aligned with the second inner bore of the pipe for flowing the lubrication oil. The multimeter can be coupled to the fuel detection film at the two locations on the fuel detection film. The two locations can be at opposite ends of the fuel detection film. The first portion and the second portion can be made of silicon, glass, quartz, polymethyl methacrylate, or any combinations of these. The fuel detection film can have a thickness in a range of from about 5 μm to about 500 μm

The details of one or more implementations of the subject matter of this disclosure are set forth in the accompanying drawings and the description. Other features, aspects, and advantages of the subject matter will become apparent from the description, the drawings, and the claims.

### DESCRIPTION OF DRAWINGS

FIG. 1A is a schematic diagram of an example assembly for an apparatus for detecting hydrocarbon fuels in lubrication oils.

FIG. 1B is a schematic diagram of the apparatus of FIG. 1A after it has been assembled.

FIG. 2 is a schematic diagram of an example system for detecting hydrocarbon fuels in lubrication oils.

FIG. 3 is a flow chart of an example method for detecting hydrocarbon fuels in lubrication oils.

FIG. 4 is a graph of an example experiment that correlates diesel concentration in lubrication oil with dissolution time of a fuel detection film.

### DETAILED DESCRIPTION

This disclosure describes a device for detecting the presence of hydrocarbon fuels in lubrication oils. The device includes patterned sheet of cyclic olefin copolymer (COC). COC is soluble in aromatic hydrocarbons, which are typically present in fuels such as gasoline and diesel. Some examples of aromatic hydrocarbons include benzene, alkylbenzenes (for example, toluene), and naphthalene. COC is insoluble in paraffins, which typically make up high viscosity lubrication oils. Paraffins are saturated hydrocarbons and often referred to as alkanes. A lubrication oil (which is being tested for the presence of hydrocarbon fuels) is flowed through the device. If hydrocarbon fuels are present in the lubrication oil, the hydrocarbon fuels will cause the COC sheet to dissolve and change shape. The dissolution rate of the COC sheet can be correlated to the concentration of hydrocarbon fuel present in the lubrication oil.

The subject matter described in this disclosure can be implemented in particular implementations, so as to realize one or more of the following advantages. The apparatus is small and portable (for example, having a total volume of less than 1 cubic feet), thus the apparatus can be used in various locations that use or transport lubrication oils. For example, the apparatus can be used in a laboratory, onsite at a pipeline, or onsite at a processing facility that uses lubrication oil. The apparatus can be used to quickly test a small sample of a lubrication oil to identify cases in which smugglers may be trying to smuggle hydrocarbon fuels by hiding them in the transport of lubrication oils. Thus, the apparatuses and methods described can be implemented to mitigate or prevent hydrocarbon fuel smuggling in a quick, reliable manner. The patterned COC sheet is porous and can dissolve quickly. The patterned COC sheet can enhance surface area contact of the test fluid with the COC sheet. The patterned COC sheet can be versatile in that it can fabricated to have varying orientations and/or patterns.

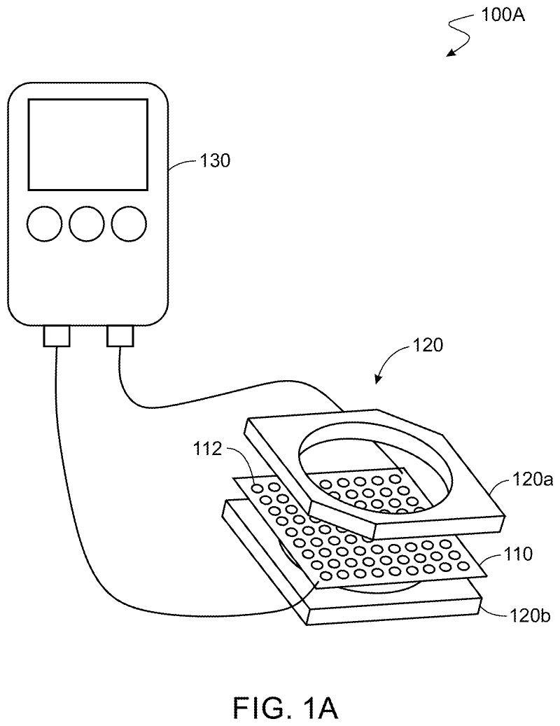

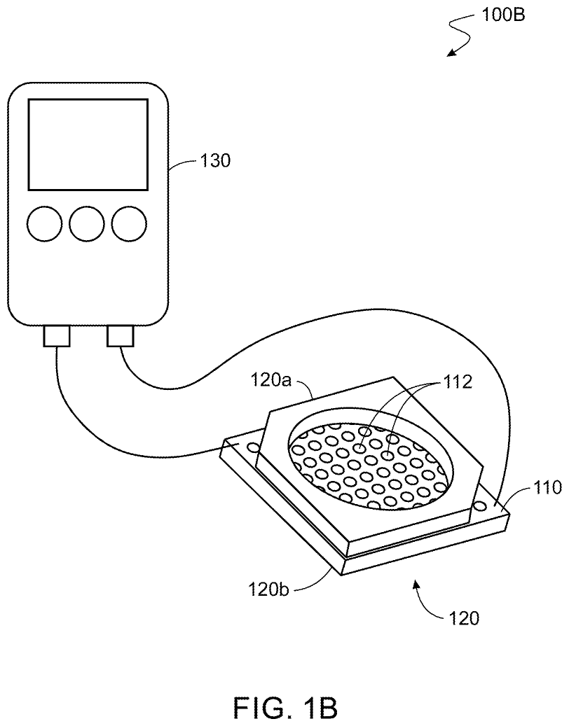

FIG. 1A is a schematic diagram of an example assembly 100A for an apparatus 100B for detecting hydrocarbon fuels in lubrication oils. FIG. 1B is a schematic diagram of the apparatus 100B after the assembly 100A of FIG. 1A has been assembled. The assembly 100A (and apparatus 100B) includes a fuel detection film 110 and a housing 120. The fuel detection film 110 includes a cyclic olefin copolymer. For example, the fuel detection film 110 is partially or wholly made of the cyclic olefin copolymer. The cyclic olefin copolymer is insoluble in acyclic (branched or unbranched) saturated hydrocarbons having a chemical formula of CₙH₂ₙ₊₂ (paraffins), where n is an integer. Some examples of acyclic saturated hydrocarbons include methane, ethane, propane, butane, pentane, and hexane (and isomers thereof). The cyclic olefin copolymer is soluble in aromatic hydrocarbons, such as benzene, alkylbenzenes (for example, toluene and xylene), styrene, and naphthalene. In some implementations, the cyclic olefin copolymer is soluble in substituted aromatic hydrocarbons (that is, aromatic hydrocarbons that include substitutions of other elements, such as oxygen or nitrogen), such as aniline, phenol, and benzaldehyde. In some implementations, the cyclic olefin copolymer includes an ethylene-norbornene copolymer. As one non-limiting example, the cyclic olefin copolymer can include TOPAS® cyclic olefin copolymer grade 5013L-10 with a 3-millimeter nominal granule size.

The fuel detection film 110 has a porous structure that defines pores 112. The pores 112 of the fuel detection film 110 are configured to allow flow of a lubrication oil through the fuel detection film 110 via the pores 112. The pores 112 can be of uniform size or can have varying sizes. The pores 112 can have the same shape or can have varying shapes. The pores 112 can be distributed uniformly across the fuel detection film 110 or can be distributed randomly across the fuel detection film 110. In some implementations, the openings of the pores 112 have an average diameter in a range of from about 10 micrometers (μm) to about 100 μm. The fuel detection film 110 can be made by spin coating. For example, the cyclic olefin copolymer can be dissolved in a solvent (such as toluene), and the solution (including solvent and dissolved cyclic olefin copolymer) can be spin-coated onto a patterned substrate, which acts as a mold. After spin coating, the solvent can then be evaporated (for example, by a heater or by resting at room temperature), leaving behind the cyclic olefin copolymer. The cyclic olefin copolymer can be released from the substrate (mold) to form the fuel detection film 110. The locations of the pores 112 of the fuel detection film 110 would have an inverse relationship with the substrate (mold), in which the locations of the pores 112 would be at the analogous locations of the raised portions of the substrate (mold). In other words, the fuel detection film 110 can be described as being a negative of the substrate (mold). In some implementations, the fuel detection film 110 has a thickness in a range of from about 5 μm to about 500 μm.

The housing 120 is configured to couple to a pipe that flows lubrication oil. The housing 120 defines a slot (122, shown in FIG. 1B with the housing 120 in assembled form) that is configured to hold the fuel detection film 110. The housing 120 is made of any suitable material that is compatible with (that is, does not react with or degrade in response to exposure to) hydrocarbons and lubrication oil. For example, the housing 120 is made of silicon, glass, quartz, polymethyl methacrylate (PMMA), or any combinations of these. In some implementations, as shown in FIG. 1A, the housing 120 includes a first portion 120a and a second portion 120b. The first portion 120a and the second portion 120b can be coupled together to form the assembled housing 120. The slot 122 is defined between the first portion 120a and the second portion 120b. In some implementations, the first portion 120a and the second portion 120b are adhered together (for example, by an adhesive). In some implementations, the first portion 120a and the second portion 120b are mechanically coupled together (for example, by a fastener, screw, bolt, or any combinations of these). In some implementations, the first portion 120a and the second portion 120b are made of the same material. In some implementations, the first portion 120a and the second portion 120b are made of different materials. In some implementations, the first portion 120a is made of silicon, glass, quartz, PMMA, or any combinations of these. In some implementations, the second portion 120b is made of silicon, glass, quartz, PMMA, or any combinations of these.

The fuel detection film 110 is configured to, while being held by the slot 122 of the housing 120 that is coupled to the pipe as the lubrication oil flows in the pipe, at least partially dissolve in a presence of an aromatic hydrocarbon in the lubrication oil flowing through the fuel detection film 110. For example, if no aromatic hydrocarbons are present in the lubrication oil (that is, hydrocarbon fuel is not present in the lubrication oil), then the fuel detection film 110 would remain intact and not dissolve in response to the lubrication oil flowing through the fuel detection film 110. As another example, if an aromatic hydrocarbon is present in the lubrication oil (that is, hydrocarbon fuel is present in the lubrication oil), then the fuel detection film 110 would at least partially dissolve in response to the lubrication oil flowing through the fuel detection film 110. Thus, the fuel detection film 110 can be implemented to identify lubrication oils that include hydrocarbon fuel.

In some implementations, the assembly 100A (and apparatus 100B) includes a multimeter 130. The multimeter 130 can be coupled to the fuel detection film 110 at two locations on the fuel detection film 110. For example, the multimeter 130 can be coupled to the fuel detection film 110 at opposite ends of the fuel detection film 110. The multimeter 130 can be configured to apply a current across the fuel detection film 110, for example, while the lubrication oil flows through the fuel detection film 110. The current exhibits a voltage drop across the fuel detection film 110. The multimeter 130 is configured to detect the voltage drop exhibited by the current applied across the fuel detection film 110. The multimeter 130 is configured to measure a resistance of the fuel detection film 110 at least based on the voltage drop exhibited by the current applied across the fuel detection film 110. A change in the resistance of the fuel detection film 110 can indicate that at least part of the fuel detection film 110 has dissolved, which in turn, can indicate the presence of an aromatic hydrocarbon (for example, hydrocarbon fuel such as gasoline and/or diesel) in the lubrication oil. For example, a decrease in resistance of the fuel detection film 110 can indicate loss of material (for example, at least partial dissolution) of the fuel detection film 110, which can indicate the presence of an aromatic hydrocarbon (for example, hydrocarbon fuel such as gasoline and/or diesel) in the lubrication oil.

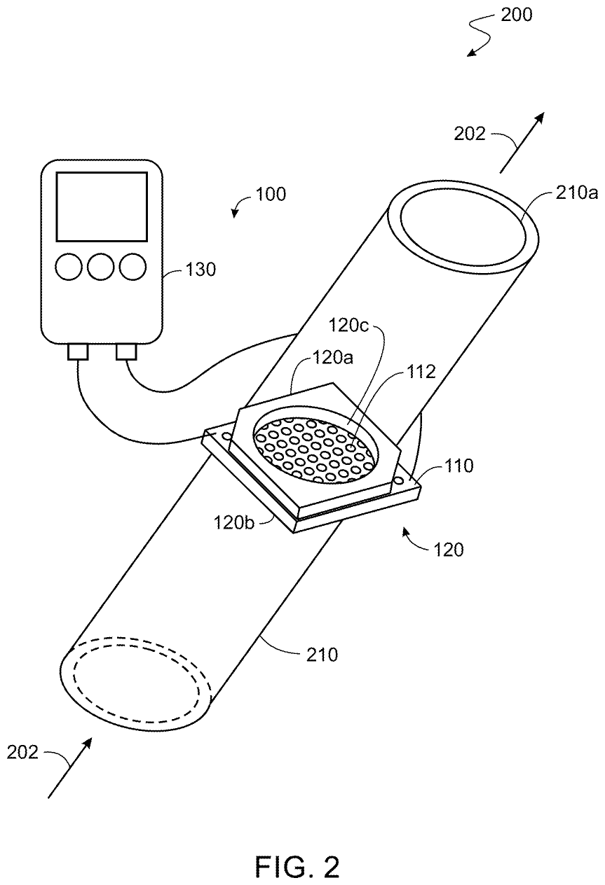

FIG. 2 is a schematic diagram of an example system 200 for detecting hydrocarbon fuels in lubrication oils. The system 200 includes a pipe 210 and the apparatus 100B. The pipe 210 defines an inner bore 210a that is configured to flow a lubrication oil 202. The housing 120 of the apparatus 100B is coupled to the pipe 210. The housing 120 defines an inner bore 120c. The inner bore 120c of the housing 120 is aligned with the inner bore 210a of the pipe 210 for flow of the lubrication oil 202. For example, a central axis of the inner bore 120c of the housing 120 can be inline with a central axis of the inner bore 210a of the pipe 210. The housing 120 defines the cross-sectional slot 122, and the fuel detection film 110 is disposed within the slot 122. The fuel detection film 110 spans across the inner bore 120c of the housing 120. The multimeter 130 is coupled to the fuel detection film 110 at two locations on the fuel detection film 110. For example, the multimeter 130 is coupled at opposite ends of the fuel detection film 110. As the lubrication oil 202 flows through the pipe 210 and across the fuel detection film 110, the multimeter 130 applies a current across the fuel detection film 110. The current applied by the multimeter 130 exhibits a voltage drop across the fuel detection film 110. The multimeter 130 detects the voltage drop exhibited by the current applied across the fuel detection film 110. The multimeter 130 measures a resistance of the fuel detection film 110 at least based on the voltage drop exhibited by the current applied across the fuel detection film 110. A decrease in the resistance of the fuel detection film 110 can indicate that at least part of the fuel detection film 110 has dissolved, which in turn, can indicate the presence of an aromatic hydrocarbon (for example, hydrocarbon fuel such as gasoline and/or diesel) in the lubrication oil 202.

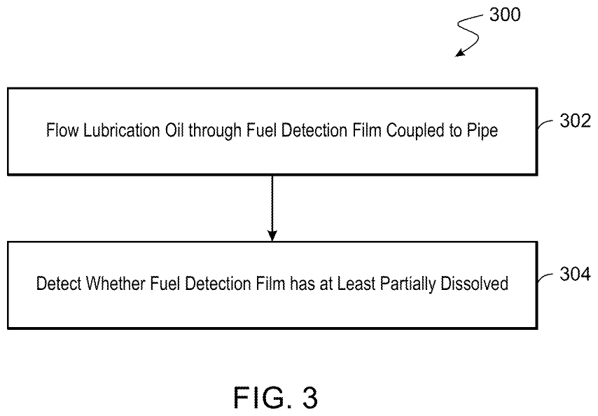

FIG. 3 is a flow chart of an example method 300 for detecting hydrocarbon fuels in lubrication oils. The apparatus 100B or the system 200 can, for example, implement the method 300. At block 302, a lubrication oil (such as the lubrication oil 202) is flowed through a fuel detection film (such as the fuel detection film 110) coupled to a pipe (such as the pipe 210). As described previously, the fuel detection film 110 includes a cyclic olefin copolymer that is insoluble in acyclic saturated hydrocarbons, and the fuel detection film 110 has a porous structure defining pores (such as the pores 112) that are configured to allow flow of the lubrication oil 202 through the fuel detection film 110 via the pores 112. The fuel detection film 110 is configured to at least partially dissolve in a presence of an aromatic hydrocarbon (for example, a component of a hydrocarbon fuel, such as gasoline and/or diesel) in the lubrication oil 202 flowing through the fuel detection film 110. At block 304, it is detected whether the fuel detection film 110 has at least partially dissolved in response to the lubrication oil 202 flowing through the fuel detection film 110. Detection at block 304 that at least a portion of the fuel detection film 110 has dissolved (at least partial dissolution of the fuel detection film 110) in response to the lubrication oil 202 flowing through the fuel detection film 110 at block 302 indicates that the aromatic hydrocarbon is present in the lubrication oil 202. Thus, detecting that at least a portion of the fuel detection film 110 has dissolved at block 304 indicates that hydrocarbon fuel (such as gasoline and/or diesel) is present in the lubrication oil 202.

In some implementations, the method 300 includes applying a current across the fuel detection film 110 as the lubrication oil 202 flows through the fuel detection film 110. The current can be applied across the fuel detection film 110, for example, by the multimeter 130. In some implementations, the method 300 includes measuring a resistance of the fuel detection film 110 at least based on a voltage drop exhibited by the current applied across the fuel detection film 110 in response to the current applied across the fuel detection film 110. The resistance of the fuel detection film 110 can be measured, for example, by the multimeter 130. Detecting whether the fuel detection film 110 has at least partially dissolved at block 304 can include detecting a change (for example, a decrease) in the resistance of the fuel detection film 110. A decrease in the resistance of the fuel detection film 110 can, for example, indicate at least partial dissolution of the fuel detection film 110 and, in turn, the presence of the aromatic hydrocarbon in the lubrication oil 202.

In some implementations, the fuel detection film 110 is disposed within a cross-section slot (such as the slot 122) of a housing (such as the housing 120) that is coupled to the pipe 210. As described previously, the housing 120 can define an inner bore 120c that is aligned with an inner bore 210a of the pipe 210 while the housing 120 is coupled to the pipe 210. The fuel detection film 110 can span across the inner bore 120c of the housing 120. The housing 120 can include a first portion 120a and a second portion 120b that are coupled together. The cross-sectional slot 122 can be defined between the first portion 120a and the second portion 120b. The fuel detection film 110 can be sandwiched between the first portion 120a and the second portion 120b. The method 300 can include disposing the fuel detection film 110 within the cross-sectional slot 122 of the housing 120 and coupling the first portion 120a and the second portion 120b together, such that the fuel detection film 110 is sandwiched between the first portion 120a and the second portion 120b, and the fuel detection film 110 spans across the inner bore 120c of the housing 120. The method 300 can include coupling the housing 120 to the pipe 210, such that the inner bore 120c of the housing 120 is aligned with the inner bore 210a of the pipe 210 for flowing the lubrication oil 202. The method 300 can include coupling the multimeter 130 to the fuel detection film 110 at opposite ends of the fuel detection film 110.

### Example

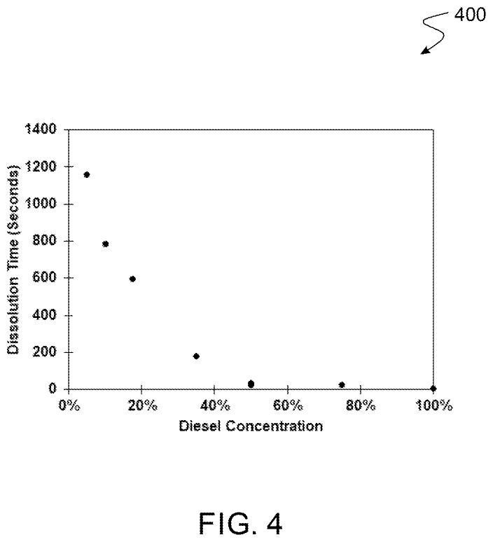

FIG. 4 is a graph 400 of an example experiment that correlates diesel concentration (x-axis) in lubrication oil with dissolution time (y-axis) of an implementation of the fuel detection film 110. Dissolution of cyclic olefin copolymer (present in the fuel detection film 110) in response to exposure to aromatic hydrocarbons (for example, present in diesel and/or gasoline) can depend on various factors, such as solvent type (aromatic, paraffin, aqueous, etc.), operating temperature, motion of molecules, concentration of aromatic hydrocarbons, and thin film thickness. In this specific experiment, the operating temperature was maintained at room temperature. The graph 400 for the experiment shows that, in general, dissolution time of the fuel detection film 110 decreased as diesel concentration increased.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

As used in this disclosure, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

As used in this disclosure, the term “about” or “approximately” can allow for a degree of variability in a value or range, for example, within 10%, within 5%, or within 1% of a stated value or of a stated limit of a range.

As used in this disclosure, the term “substantially” refers to a majority of, or mostly, as in at least about 50%, 60%, 70%, 80%, 90%, 95%, 96%, 97%, 98%, 99%, 99.5%, 99.9%, 99.99%, or at least about 99.999% or more.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “0.1% to about 5%” or “0.1% to 5%” should be interpreted to include about 0.1% to about 5%, as well as the individual values (for example, 1%, 2%, 3%, and 4%) and the sub-ranges (for example, 0.1% to 0.5%, 1.1% to 2.2%, 3.3% to 4.4%) within the indicated range. The statement “X to Y” has the same meaning as “about X to about Y,” unless indicated otherwise. Likewise, the statement “X, Y, or Z” has the same meaning as “about X, about Y, or about Z,” unless indicated otherwise.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described components and systems can generally be integrated together or packaged into multiple products.

Accordingly, the previously described example implementations do not define or constrain the present disclosure. Other changes, substitutions, and alterations are also possible without departing from the spirit and scope of the present disclosure.

What is claimed is:

1. An assembly comprising:
a fuel detection film comprising a cyclic olefin copolymer that is insoluble in acyclic saturated hydrocarbons, the fuel detection film having a porous structure defining a plurality of pores configured to allow flow of a lubrication oil through the fuel detection film via the plurality of pores; and
a housing configured to couple to a pipe flowing the lubrication oil, the housing defining a slot configured to hold the fuel detection film, wherein the fuel detection film, while held by the slot of the housing coupled to the pipe as the lubrication oil flows in the pipe, is configured to at least partially dissolve in a presence of an aromatic hydrocarbon in the lubrication oil flowing through the fuel detection film.

2. The assembly of claim 1, comprising a multimeter coupled to the fuel detection film at two locations on the fuel detection film, the multimeter configured to apply a current across the fuel detection film, the multimeter configured to measure a resistance of the fuel detection film at least based on a voltage drop exhibited by the current applied across the fuel detection film in response to the current applied across the fuel detection film, wherein a decrease in the resistance of the fuel detection film indicates at least partial dissolution of the fuel detection film and the presence of the aromatic hydrocarbon in the lubrication oil.

3. The assembly of claim 2, wherein the housing comprises a first portion and a second portion coupled together, and the slot is defined between the first portion and the second portion.

4. The assembly of claim 3, wherein the first portion and the second portion are made of silicon, glass, quartz, polymethyl methacrylate, or any combinations thereof.

5. The assembly of claim 4, wherein the fuel detection film has a thickness in a range of from about 5 micrometers (μm) to about 500 μm.

6. A system comprising:
a pipe defining a first inner bore configured to flow a lubrication oil;
a housing coupled to the pipe, the housing defining a second inner bore and a cross-sectional slot, the second inner bore of the housing aligned with the first inner bore of the pipe for flow of the lubrication oil;
a fuel detection film disposed within the cross-sectional slot of the housing, the fuel detection film spanning across the second inner bore of the housing, the fuel detection film comprising a cyclic olefin copolymer that is insoluble in acyclic saturated hydrocarbons, the fuel detection film having a porous structure defining a plurality of pores configured to allow flow of the lubrication oil through the fuel detection film via the plurality of pores, the fuel detection film is configured to at least partially dissolve in a presence of an aromatic hydrocarbon in the lubrication oil flowing through the fuel detection film; and
a multimeter coupled to the fuel detection film at two locations on the fuel detection film, the multimeter configured to apply a current across the fuel detection film, the multimeter configured to measure a resistance of the fuel detection film at least based on a voltage drop exhibited by the current applied across the fuel detection film, wherein a change in the resistance of the fuel detection film indicates at least partial dissolution of the fuel detection film and the presence of the aromatic hydrocarbon in the lubrication oil.

7. The system of claim 6, wherein the housing comprises a first portion and a second portion coupled together, and the cross-sectional slot is defined between the first portion and the second portion.

8. The system of claim 7, wherein the first portion and the second portion are made of silicon, glass, quartz, polymethyl methacrylate, or any combinations thereof.

9. The system of claim 8, wherein the fuel detection film has a thickness in a range of from about 5 micrometers (μm) to about 500 μm.

10. A method comprising:
flowing a lubrication oil through a fuel detection film coupled to a pipe, the fuel detection film comprising a cyclic olefin copolymer that is insoluble in acyclic saturated hydrocarbons, the fuel detection film having a porous structure defining a plurality of pores configured to allow flow of the lubrication oil through the fuel detection film via the plurality of pores, the fuel detection film configured to at least partially dissolve in a presence of an aromatic hydrocarbon in the lubrication oil flowing through the fuel detection film; and
detecting whether the fuel detection film has at least partially dissolved in response to the lubrication oil flowing through the fuel detection film, wherein at least partial dissolution of the fuel detection film in response to the lubrication oil flowing through the fuel detection film indicates the presence of an aromatic hydrocarbon in the lubrication oil.

11. The method of claim 10, comprising:
applying a current across the fuel detection film as the lubrication oil flows through the fuel detection film; and
measuring a resistance of the fuel detection film at least based on a voltage drop exhibited by the current applied across the fuel detection film in response to the current applied across the fuel detection film.

12. The method of claim 11, wherein a multimeter coupled to the fuel detection film at two locations on the fuel detection film applies the current across the fuel detection film and measures the resistance of the fuel detection film, wherein detecting whether the fuel detection film has at least partially dissolved comprises detecting a change in the resistance of the fuel detection film, wherein a decrease in the resistance of the fuel detection film indicates at least partial dissolution of the fuel detection film and the presence of the aromatic hydrocarbon in the lubrication oil.

13. The method of claim 12, wherein:
the fuel detection film is disposed within a cross-sectional slot of a housing that is coupled to the pipe;
the housing defines a first inner bore that is aligned with a second inner bore of the pipe through which the lubrication oil flows; and
the fuel detection film spans across the first inner bore of the housing.

14. The method of claim 13, wherein the housing comprises a first portion and a second portion coupled together, the cross-sectional slot defined between the first portion and the second portion, and the fuel detection film is sandwiched between the first portion and the second portion.

15. The method of claim 14, comprising disposing the fuel detection film within the cross-sectional slot of the housing and coupling the first portion and the second portion together, such that the fuel detection film is sandwiched between the first portion and the second portion, and the fuel detection film spans across the first inner bore of the housing.

16. The method of claim 15, comprising coupling the housing to the pipe, such that the first inner bore of the housing is aligned with the second inner bore of the pipe for flowing the lubrication oil.

17. The method of claim 16, comprising coupling the multimeter to the fuel detection film at the two locations on the fuel detection film, wherein the two locations are at opposite ends of the fuel detection film.

18. The method of claim 17, wherein the first portion and the second portion are made of silicon, glass, quartz, polymethyl methacrylate, or any combinations thereof.

19. The method of claim 18, wherein the fuel detection film has a thickness in a range of from about 5 micrometers (μm) to about 500 μm.
